(19) United States
(12) Patent Application Publication
(10) Pub. No.: US 2026/0103525 A1
(43) Pub. Date: Apr. 16, 2026

(54) KLRG1 SIGNALLING THERAPY FOR INFECTIOUS DISEASE

(71) Applicant: The Brigham and Women's Hospital, Inc., Boston, MA (US)

(72) Inventor: Steven GREENBERG, Watertown, MA (US)

(21) Appl. No.: 19/115,326

(22) PCT Filed: Sep. 27, 2023
(86) PCT No.: PCT/US2023/033833

§ 371 Date: Mar. 26, 2025

Related U.S. Application Data
(60) Provisional application No. 63/410,301, filed on Sep. 27, 2022.

(51) Int. Cl. C07K 16/28; A61K 31/343; A61K 31/427; A61K 31/4418; A61K 31/4439; A61K 31/505; A61K 31/513; A61K 31/52; A61K 31/536; A61K 31/675; A61K 31/7068; A61K 31/7072; A61K 39/00; A61K 39/21; A61K 39/395; A61P 31/18; C12N 15/113

(52) U.S. Cl. CPC C07K 16/2851; A61K 31/343; A61K 31/427; A61K 31/4418; A61K 31/4439; A61K 31/505; A61K 31/513; A61K 31/52; A61K 31/536; A61K 31/675; A61K 31/7068; A61K 31/7072; A61K 39/21; A61K 39/3955; A61P 31/18; C12N 15/1138; A61K 2039/505; C07K 2317/76; C12N 2310/11; C12N 2310/14

(57) ABSTRACT

Disclosed herein are methods for of preventing a human immunodeficiency virus (HIV) infection in a subject or treating a subject suffering from an infectious disease caused by HIV, comprising administering to the subject an effective amount of a killer cell lectin-like receptor G1 (KLRG1) antagonist. The methods can further comprise administering other therapies to the subject, such as an anti-viral therapy.

### BACKGROUND

Lymphocyte co-inhibitory receptors modulate the action of the adaptive immune system, for example T cells and NK cells, in response to activating signals such as antigenic peptides in the context of the major histocompatibility complex (MHC) binding to the T cell receptor (TCR). Co-inhibitory receptors include PD-1, LAG-3, TIM-3, and CTLA4. The action of co-inhibitory receptors is generally carried out by binding of a ligand to the extracellular domain of the co-inhibitory receptor followed by recruitment of intracellular phosphatases by an immunoreceptor tyrosine-based inhibition motif (ITIM) located in the intracellular domain of the co-inhibitory receptor. The action of co-inhibitory receptors is generally to dampen the immune response of TCR engagement. In recent years it has been shown that agents that block the activity of co-inhibitory receptors can be used to treat infectious diseases.

Killer cell lectin-like receptor G1 (KLRG1) is a type II transmembrane protein acting as a co-inhibitory receptor by modulating the activity of T and NK cells. Its extracellular portion contains a C-type lectin domain whose known ligands are cadherins and its intracellular portion contains an immunoreceptor tyrosine-based inhibitory motif (ITIM) domain responsible for co-inhibition of T cell receptor (TCR) mediated signaling. KLRG1 ligands can be E-cadherin, N-cadherin, R-cadherin, or a combination thereof.

The KLRG1 receptor is expressed on T and NK cells which binds to ligands on epithelial and mesenchymal cells. The ligands for KLRG1 have been described to be E-cadherin, N-cadherin and R-cadherin.

In humans, KLRG1 expression is confined to cells of the immune system, including CD8 positive T cells, NK cells, and to a lesser extent, to CD4 positive T cells and type 2 innate lymphoid cells (ILC2s). KLRG1 expression has been associated with the late differentiated phenotype. As antigen specific T cells differentiate, they acquire increased expression of cytotoxic molecules and therefore have increased cytotoxic potential. The biological function of KLRG1 is to inhibit cytotoxicity and proliferation of these T cells. In infectious disease, it has been shown to be beneficial to restore T cell activity.

In general, a need exists to provide safe and effective therapeutic methods for preventing and treating infectious diseases through restoration of T cell activity.

### SUMMARY OF THE INVENTION

This disclosure relates to methods of treating or preventing an infectious disease in a subject, comprising administering to the subject an effective amount of a killer cell lectin-like receptor G1 (KLRG1) antagonist.

In some embodiments, the KLRG1 antagonist can be an antibody or an antigen-binding fragment thereof that (i) specifically binds killer cell lectin-like receptor G1 (KLRG1); and (ii) disrupts KLRG1 signaling with a ligand of KLRG1 selected from the group consisting of E-cadherin, N-cadherin, and R-cadherin.

In some embodiments, the antibody or the antigen-binding fragment can comprise a CDR-H1 selected from the group consisting of SEQ ID NO: 9, SEQ ID NO: 1, and SEQ ID NO: 16; a CDR-H2 selected from the group consisting of SEQ ID NO: 10, SEQ ID NO: 2, and SEQ ID NO: 17; a CDR-H3 selected from the group consisting of SEQ ID NO: 11, SEQ ID NO: 3, and SEQ ID NO: 18; a CDR-L1 selected from the group consisting of SEQ ID NO: 12, SEQ ID NO: 4, and SEQ ID NO: 19; a CDR-L2 selected from the group consisting of SEQ ID NO: 5 and SEQ ID NO: 20; and a CDR-L3 selected from the group consisting of SEQ ID NO: 13, SEQ ID NO: 6, and SEQ ID NO: 21.

In some embodiments, the antibody or the antigen-binding fragment can comprise the CDR-H1 of SEQ ID NO: 9, the CDR-H2 of SEQ ID NO: 10, the CDR-H3 of SEQ ID NO: 11, the CDR-L1 of SEQ ID NO: 12, the CDR-L2 of SEQ ID NO: 5, and the CDR-L3 of SEQ ID NO: 13; the CDR-H1 of SEQ ID NO: 1, the CDR-H2 of SEQ ID NO: 2, the CDR-H3 of SEQ ID NO: 3, the CDR-L1 of SEQ ID NO: 4, the CDR-L2 of SEQ ID NO: 5, and the CDR-L3 of SEQ ID NO: 6; the CDR-H1 of SEQ ID NO: 16, the CDR-H2 of SEQ ID NO: 17, the CDR-H3 of SEQ ID NO: 18, the CDR-L1 of SEQ ID NO: 19, the CDR-L2 of SEQ ID NO: 20, and the CDR-L3 of SEQ ID NO: 21; the CDR-H1 of SEQ ID NO: 9, the CDR-H2 of SEQ ID NO: 10, the CDR-H3 of SEQ ID NO: 11, the CDR-L1 of SEQ ID NO: 12, the CDR-L2 of SEQ ID NO: 5, and the CDR-L3 of SEQ ID NO: 13; or the CDR-H1 of SEQ ID NO: 9, the CDR-H2 of SEQ ID NO: 10, the CDR-H3 of SEQ ID NO: 11, the CDR-L1 of SEQ ID NO: 26, the CDR-L2 of SEQ ID NO: 5, and the CDR-L3 of SEQ ID NO: 6.

In some embodiments, the antibody or the antigen-binding fragment can comprise a heavy chain variable region with at least 90% identity to SEQ ID NO: 14 and a light chain variable region with at least 90% identity to SEQ ID NO: 15; a heavy chain variable region with at least 90% identity to SEQ ID NO: 7 and a light chain variable region with at least 90% identity to SEQ ID NO: 8; a heavy chain variable region with at least 90% identity to SEQ ID NO: 22 and a light chain variable region with at least 90% identity to SEQ ID NO: 23; a heavy chain variable region with at least 90% identity to SEQ ID NO: 24 and a light chain variable region with at least 90% identity to SEQ ID NO: 25; or a heavy chain variable region with at least 90% identity to SEQ ID NO: 14 and a light chain variable region with at least 90% identity to SEQ ID NO: 27.

In a particular embodiment, the antibody or the antigen-binding fragment can comprise SEQ ID NO: 14 and SEQ ID NO: 15.

In the method, the infectious disease can be caused by a bacterium or a virus. In some embodiments, the infectious disease can be caused by Mycobacterium tuberculosis. In some embodiments, the infectious disease can be caused by hepatitis B virus, Epstein-Barr virus (EBV), cytomegalovirus, human herpesvirus 3 (HHV3), or human immunodeficiency virus (HIV).

In a particular embodiment, the infectious disease can be caused by HIV.

The method can further comprise administering to the subject a non-KLRG1-antagonist therapy for the infectious disease. For example, the infectious disease can be tuberculosis and the non-KLRG1-antagonist therapy can be selected from the group consisting of isoniazid, rifampin, ethambutol, pyrazinamide, fluoroquinolones, amikacin, capreomycin, bedaquiline, linezolid, and combinations of thereof; the infectious disease can be hepatitis B and the non-KLRG1-antagonist therapy can be selected from the group consisting of entecavir, tenofovir, lamivudine, adefovir, telbivudine, interferon alfa-2b, liver transplantation, and combinations thereof; the infectious disease can be chickenpox or shingles and the non-KLRG1-antagonist therapy can be acyclovir, valacyclovir, famciclovir, and combinations thereof; or the infectious disease can be AIDS and the non-KLRG1-antagonist therapy can be selected from the group consisting of efavirenz, rilpivirine, doravirine, abacavir, tenofovir disoproxil fumarate, emtricitabine, lamivudine, zidovudine, emtricitabine/tenofovir disoproxil fumarate, emtricitabine/tenofovir alafenamide fumarate, atazanavir, darunavir, lopinavir/ritonavir, bictegravir sodium/emtricitabine/tenofovir alafenamide fumarate, raltegravir, dolutegravir, cabotegravir, enfuvirtide, maraviroc, and combinations thereof.

In some embodiments, the non-KLRG1-antagonist therapy can be a vaccine therapy. For example, the infectious disease can be tuberculosis and the vaccine therapy can be bacille Calmette-Guerin (BCG); the infectious disease can be hepatitis B and the vaccine therapy can be a recombinant hepatitis B vaccine, such as Engerix-B (GSK plc, Brentford, United Kingdom) or Recombivax-HB (Merck and Co., Inc., Rahway, NJ); the infectious disease can be chickenpox and the vaccine therapy can be a varicella vaccine; or the infectious disease can be shingles and the vaccine therapy can be an adjuvanted recombinant zoster vaccine, such as Shingrix (GSK plc), or a live zoster vaccine, such as Zostavax (Merck and Co., Inc.).

The disclosure also relates to a method of treating a subject suffering from AIDS, comprising administering to the subject an effective amount of an antibody or an antigen-binding fragment thereof that (i) specifically binds killer cell lectin-like receptor G1 (KLRG1); (ii) disrupts KLRG1 signaling with a ligand of KLRG1 selected from the group consisting of E-cadherin, N-cadherin, and R-cadherin; and (iii) comprises a CDR-H1 having SEQ ID NO: 9, a CDR-H2 having SEQ ID NO: 10, a CDR-H3 having SEQ ID NO: 11, a CDR-L1 having SEQ ID NO: 12, a CDR-L2 having SEQ ID NO: 5, and a CDR-L3 having SEQ ID NO: 13.

In one aspect, the disclosure relates to method of preventing a human immunodeficiency virus (HIV) infection in a subject or treating a subject suffering from an infectious disease caused by HIV, comprising administering to the subject an effective amount of an antibody or an antigen-binding fragment thereof that:
(i) specifically binds killer cell lectin-like receptor G1 (KLRG1); and
(ii) comprises a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 12, a CDR-L2 comprising the sequence of SEQ ID NO: 5, and a CDR-L3 comprising the sequence of SEQ ID NO: 13;
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 28, a CDR-L2 comprising the sequence of SEQ ID NO: 5, and a CDR-L3 comprising the sequence of SEQ ID NO: 13;
a CDR-H1 comprising the sequence of SEQ ID NO: 1, a CDR-H2 comprising the sequence of SEQ ID NO: 2, a CDR-H3 comprising the sequence of SEQ ID NO: 3, a CDR-L1 comprising the sequence of SEQ ID NO: 4, a CDR-L2 comprising the sequence of SEQ ID NO: 5, and a CDR-L3 comprising the sequence of SEQ ID NO: 6;
a CDR-H1 comprising the sequence of SEQ ID NO: 16, a CDR-H2 comprising the sequence of SEQ ID NO: 17, a CDR-H3 comprising the sequence of SEQ ID NO: 18, a CDR-L1 comprising the sequence of SEQ ID NO: 19, a CDR-L2 comprising the sequence of SEQ ID NO: 20, and a CDR-L3 comprising the sequence of SEQ ID NO: 21; or
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 26, a CDR-L2 comprising the sequence of SEQ ID NO: 5, and a CDR-L3 comprising the sequence of SEQ ID NO: 6.

In some embodiments, the CDR-H1 comprises the sequence of SEQ ID NO: 9, the CDR-H2 comprises the sequence of SEQ ID NO: 10, the CDR-H3 comprises the sequence of SEQ ID NO: 11, the CDR-L1 comprises the sequence of SEQ ID NO: 12, the CDR-L2 comprises the sequence of SEQ ID NO: 5, and the CDR-L3 comprises the sequence of SEQ ID NO: 13.

In other embodiments, the antibody or the antigen-binding fragment comprises:
a heavy chain variable region with at least 90% identity to the sequence of SEQ ID NO: 24 and a light chain variable region with at least 90% identity to the sequence of SEQ ID NO: 25;
a heavy chain variable region with at least 90% identity to the sequence of SEQ ID NO: 14 and a light chain variable region with at least 90% identity to the sequence of SEQ ID NO: 15;
a heavy chain variable region with at least 90% identity to the sequence of SEQ ID NO: 14 and a light chain variable region with at least 90% identity to the sequence of SEQ ID NO: 29;
a heavy chain variable region with at least 90% identity to the sequence of SEQ ID NO: 7 and a light chain variable region with at least 90% identity to the sequence of SEQ ID NO: 8;
a heavy chain variable region with at least 90% identity to the sequence of SEQ ID NO: 22 and a light chain variable region with at least 90% identity to the sequence of SEQ ID NO: 23; or
a heavy chain variable region with at least 90% identity to the sequence of SEQ ID NO: 14 and a light chain variable region with at least 90% identity to the sequence of SEQ ID NO: 27.

In one embodiment, the antibody or the antigen-binding fragment comprises the sequences of SEQ ID NO: 24 and SEQ ID NO: 25.

In another aspect, the disclosure relates to a method of treating a subject suffering from AIDS, comprising administering to the subject an effective amount of an antibody or an antigen-binding fragment thereof that:
(i) specifically binds killer cell lectin-like receptor G1 (KLRG1); and
(ii) comprises a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 12, a CDR-L2 comprising the sequence of SEQ ID NO: 5, and a CDR-L3 comprising the sequence of SEQ ID NO: 13;
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 28, a CDR-L2 comprising the sequence of SEQ ID NO: 5, and a CDR-L3 comprising the sequence of SEQ ID NO: 13;
a CDR-H1 comprising the sequence of SEQ ID NO: 1, a CDR-H2 comprising the sequence of SEQ ID NO: 2, a CDR-H3 comprising the sequence of SEQ ID NO: 3, a CDR-L1 comprising the sequence of SEQ ID NO: 4, a CDR-L2 comprising the sequence of SEQ ID NO: 5, and a CDR-L3 comprising the sequence of SEQ ID NO: 6;
a CDR-H1 comprising the sequence of SEQ ID NO: 16, a CDR-H2 comprising the sequence of SEQ ID NO: 17, a CDR-H3 comprising the sequence of SEQ ID NO: 18, a CDR-L1 comprising the sequence of SEQ ID NO: 19, a CDR-L2 comprising the sequence of SEQ ID NO: 20, and a CDR-L3 comprising the sequence of SEQ ID NO: 21; or
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 26, a CDR-L2 comprising the sequence of SEQ ID NO: 5, and a CDR-L3 comprising the sequence of SEQ ID NO: 6.

In an additional aspect, the disclosure relates to a method of preventing an HIV infection in a subject or treating a subject suffering from an infectious disease caused by HIV, comprising administering to the subject an effective amount of a nucleic acid agent that suppresses transcription, translation, or both of KLRG1.

In one embodiment, the nucleic acid agent is an RNAi agent.

In another embodiment, the nucleic acid agent is an antisense oligonucleotide.

In some embodiments, the antibody or antigen-binding fragment thereof or the nucleic acid agent disrupts KLRG1 signaling with a ligand of KLRG1 selected from the group consisting of E-cadherin, N-cadherin, and R-cadherin.

In another embodiment, the method prevents HIV infection in the subject.

In an additional embodiment, the method treats HIV infection in the subject.

In further embodiments, the method comprises administering to the subject a non-KLRG1-antagonist therapy for HIV infection (e.g., a non-KLRG1-antagonist therapy is selected from the group consisting of efavirenz, rilpivirine, doravirine, abacavir, tenofovir disoproxil fumarate, emtricitabine, lamivudine, zidovudine, emtricitabine/tenofovir disoproxil fumarate, emtricitabine/tenofovir alafenamide fumarate, atazanavir, darunavir, lopinavir/ritonavir, bictegravir sodium/emtricitabine/tenofovir alafenamide fumarate, raltegravir, dolutegravir, cabotegravir, enfuvirtide, maraviroc, and combinations thereof).

In another embodiment, the non-KLRG1-antagonist therapy is a vaccine therapy.

### BRIEF DESCRIPTION OF THE DRAWINGS

FIG. 1 shows KLRG1 expression in subjects with tuberculosis is increased compared to healthy subjects and normalizes with disease treatment.

FIG. 2 shows KLRG1 expression in a first cohort of subjects vaccinated with bacille Calmette-Guerin (BCG) against tuberculosis increased on average roughly 1.5-fold compared to baseline for 168 days after vaccination.

FIG. 3 shows KLRG1 expression in a second cohort of subjects vaccinated with BCG against tuberculosis increased from baseline in 7 or 9 subjects after 29 days.

FIG. 4 shows KLRG1 expression in subjects with chronic hepatitis B is increased compared to subjects with resolved or acute infection.

FIG. 5 shows KLRG1 expression in peripheral blood mononuclear cells (PBMCs) directed against Epstein-Barr virus (EBV), compared to naïve T cells.

FIG. 6 shows KLRG1 expression in CD4+ T cells directed against cytomegalovirus (CMV), compared to CD4+ T cells directed against other infectious diseases (tetanus and candida).

FIG. 7 shows KLRG1 expression in HIV-specific CD8+ T cells is higher in subjects with disease progression relative to subjects with controlled disease.

### DETAILED DESCRIPTION

This disclosure relates to methods of treating or preventing infectious diseases comprising administering to a subject in need thereof an effective amount of a KLRG1 antagonist, such as an anti-KLRG1 antibody or antigen-binding fragment.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others), and the like. In some embodiments, the mammal is a mouse. In some embodiments, the mammal is a human.

Additional description of the methods and guidance for the practice of the methods are provided herein.

### A. Methods of Treating or Preventing an Infectious Disease in a Subject

In some embodiments, the disclosure relates to a method of treating or preventing an infectious disease in a subject. The method comprises administering to the subject an effective amount of a killer cell lectin-like receptor G1 (KLRG1) antagonist, thereby treating or preventing the infectious disease in the subject.

In some embodiments of the method, preventing the infectious disease can involve administering a vaccine therapy in combination with the KLRG1 antagonist. Though not to be bound by theory, the KLRG1 antagonist may enhance responses of the subject's immune system to the vaccine, potentially leading to a more effective immune response than would be observed for the vaccine alone.

In some embodiments, the KLRG1 antagonist can be an anti-KLRG1 antibody or antigen-binding fragment, such as those described herein.

Generally, an effective amount of a KLRG1 antagonist may vary with the subject's age, condition, and sex, as well as the severity of the infectious disease, whether the disease is chronic or acute, and/or the species of infectious agent, among other parameters that will be apparent to a person of ordinary skill in the art having the benefit of the present disclosure.

For antibodies and antigen-binding fragments, an effective amount can range from about 0.001 to about 30 mg/kg body weight, preferably from about 0.01 to about 25 mg/kg body weight, from about 0.1 to about 20 mg/kg body weight, or from about 1 to about 10 mg/kg. The dosage can be adjusted, as necessary, to suit observed effects of the treatment. The appropriate dose can be chosen based on clinical indications by a treating physician.

The antibody or antigen-binding fragment can be given as a bolus dose, to maximize the circulating levels of antibody/antigen-binding fragment for the greatest length of time after the dose. Continuous infusion may also be used after the bolus dose.

In some embodiments, the KLRG1 antagonist can be an RNAi agent.

In some embodiments, the KLRG1 antagonist can be an antisense oligonucleotide.

The infectious disease can be caused by any organism, such as a virus, a bacterium, a fungus, a protozoan, or a parasite. In some embodiments, the infectious disease can be caused by a bacterium or a virus.

In some embodiments, the infectious disease can be caused by Mycobacterium tuberculosis.

In some embodiments, the infectious disease can be caused by hepatitis B virus, Epstein-Barr virus (EBV), cytomegalovirus, human herpesvirus 3 (HHV3), or human immunodeficiency virus (HIV). In some particular embodiments, the infectious disease can be caused by HIV.

In some particular embodiments, the subject can suffer from AIDS, and the KLRG1 antagonist can be an antibody or an antigen-binding fragment thereof that (i) specifically binds killer cell lectin-like receptor G1 (KLRG1); (ii) disrupts KLRG1 signaling with a ligand of KLRG1 selected from the group consisting of E-cadherin, N-cadherin, and R-cadherin; and (iii) comprises a CDR-H1 having SEQ ID NO: 9, a CDR-H2 having SEQ ID NO: 10, a CDR-H3 having SEQ ID NO: 11, a CDR-L1 having SEQ ID NO: 12, a CDR-L2 having SEQ ID NO: 5, and a CDR-L3 having SEQ ID NO: 13.

B. Expression of KLRG1 by Immune Cells of Subjects Suffering from Infectious Diseases

Increases in KLRG1 expression occur in a variety of immune cells in subjects suffering from a variety of infectious diseases. Modulation of the cytotoxic (or CD8+) T and NK cell activation involved in these disorders can be accomplished by manipulation of the KLRG1 pathway. For example, administering a KLRG1 antagonist can at least in part disrupt the interaction between KLRG1 and one or more cadherins. Though not wishing to be bound by theory, a blockade of KLRG1/E-cadherin interaction with a KLRG1 antagonist, such as antagonizing anti-KLRG1 antibodies or antigen-binding fragments, can lead to enhanced T cell proliferative responses and IFNγ secretion by these cells, consistent with a downregulatory role for the KLRG1 pathway in cytotoxic T and NK cell activation. Enhanced T cell proliferation and IFNγ secretion may lead to improved treatment of an infectious disease.

KLRG1 antagonists can treat infectious diseases in one or more manners. In some embodiments, KLRG1 antagonists can treat an acute infection. In some embodiments, KLRG1 antagonists can treat a chronic infection. In some embodiments, KLRG1 antagonists can enhance the effectiveness of vaccine therapies for the infectious disease.

KLRG1 antagonists that can be used to treat infectious diseases are described herein.

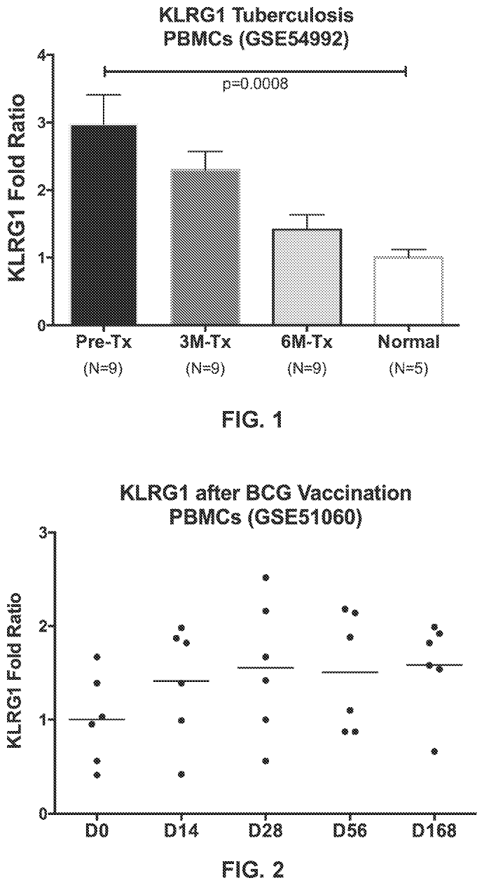

Analysis of previously unanalyzed data demonstrates the increased expression of KLRG1 on peripheral blood mononuclear cells (PBMCs) in patients with untreated tuberculosis, followed by progressive normalization of KLRG1 levels during treatment, indicating that KLRG1 is a biomarker that tracks tuberculosis disease activity. (FIG. 1, see also Example 1).

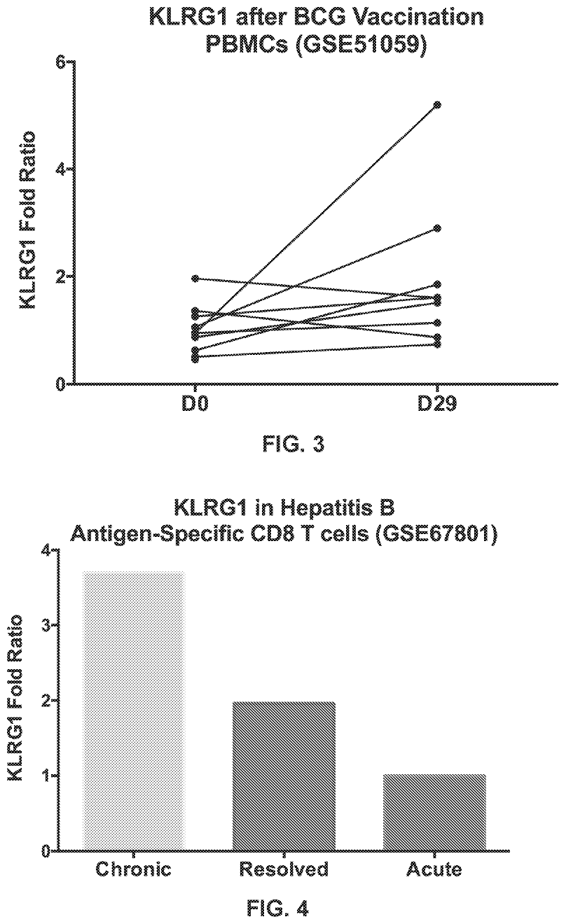

Analysis of previously unanalyzed data demonstrates increased expression of KLRG1 on PBMCs after bacille Calmette-Guerin (BCG) vaccination against tuberculosis. As KLRG1 is an inhibitory receptor, it is expected that this increased KLRG1 expression limits the effectiveness of the vaccine response. (FIGS. 2 and 3, see also Examples 2 and 3).

Analysis of previously unanalyzed data demonstrates increased expression of KLRG1 on antigen-specific CD8+ T cells in chronic hepatitis B infection, compared to resolved infection and acute infection. As KLRG1 is an inhibitory receptor, it is expected that this increased KLRG1 expression limits the effectiveness of the immune response against hepatitis B. (FIG. 4, see also Example 4).

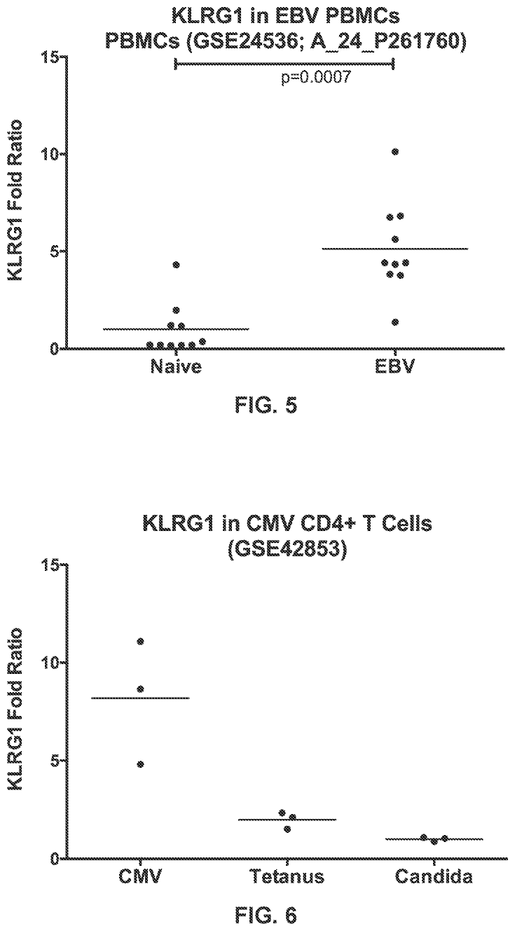

Analysis of previously unanalyzed data demonstrates increased expression of KLRG1 on antigen-specific CD8+ T cells in chronic Epstein-Barr virus infection. As KLRG1 is an inhibitory receptor, it is expected that this increased KLRG1 expression limits the effectiveness of the immune response against Epstein-Barr virus. (FIG. 5, see also Example 5).

Analysis of previously unanalyzed data demonstrates increased KLRG1 expression in CD4+ T cells directed against cytomegalovirus (CMV), compared to CD4+ T cells directed against other infectious diseases (tetanus and candida). (FIG. 6, see also Example 6).

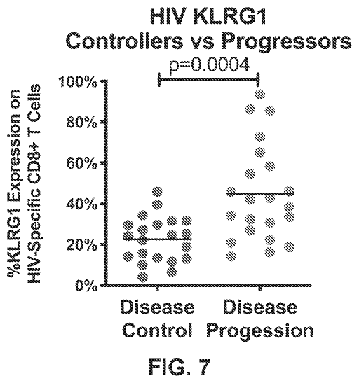

Analysis of previously unanalyzed data demonstrates increased KLRG1 expression in HIV-specific CD8+ T cells is higher in subjects with disease progression relative to subjects with controlled disease. (FIG. 7, see also Example 7).

### C. Antibodies and Antigen-Binding Fragments as KLRG1 Antagonists

In some embodiments, the disclosure provides anti-KLRG1 antibodies and antigen-binding fragments that can be used to treat infectious diseases. Preferably, the anti-KLRG1 antibodies and antigen-binding fragments can (i) specifically bind KLRG1 and (ii) disrupt KLRG1 signaling with a ligand of KLRG1 selected from the group consisting of E-cadherin, N-cadherin, and R-cadherin.

In general, antibodies can be made, for example, using traditional hybridoma techniques (Kohler and Milstein (1975) Nature, 256:495-499), recombinant DNA methods (U.S. Pat. No. 4,816,567), or phage display performed with antibody libraries (Clackson et al. (1991) Nature, 352:624-628; Marks et al. (1991) J. Mol. Biol., 222:581-597). For other antibody production techniques, see also Antibodies: A Laboratory Manual, eds. Harlow et al., Cold Spring Harbor Laboratory, 1988. The invention is not limited to any particular source, species of origin, or method of production.

Intact antibodies, also known as immunoglobulins, are typically tetrameric glycosylated proteins composed of two light (L) chains of approximately 25 kDa each and two heavy (H) chains of approximately 50 kDa each. Two types of light chain, designated as the A chain and the k chain, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2.

The subunit structures and three-dimensional configurations of different classes of immunoglobulins are well known in the art. For a review of antibody structure, see Harlow et al., supra. Briefly, each light chain is composed of an N-terminal variable domain (VL) and a constant domain (CL). Each heavy chain is composed of an N-terminal variable domain (VH), three or four constant domains (CH), and a hinge region. The CH domain most proximal to VH is designated as CH1. The VH and VL domains consist of four regions of relatively conserved sequence called framework regions (FR1, FR2, FR3, and FR4), which form a scaffold for three regions of hypervariable sequence called complementarity determining regions (CDRs). The CDRs contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L1, L2, and L3, accordingly. CDR3 and, particularly H3, are the greatest source of molecular diversity within the antigen-binding domain. H3, for example, can be as short as two amino acid residues or greater than 26.

The Fab fragment (Fragment antigen-binding) consists of the VH-CH1 and VL-CL domains covalently linked by a disulfide bond between the constant regions. To overcome the tendency of non-covalently linked VH and VL domains in the Fv to dissociate when co-expressed in a host cell, a so-called single chain (sc) Fv fragment (scFv) can be constructed. In a scFv, a flexible and adequately long polypeptide links either the C-terminus of the VH to the N-terminus of the VL or the C-terminus of the VL to the N-terminus of the VH. Most commonly, a 15-residue (Gly4Ser) 3 peptide is used as a linker but other linkers are also known in the art.

Antibody diversity is a result of combinatorial assembly of multiple germline genes encoding variable regions and a variety of somatic events. The somatic events include recombination of variable gene segments with diversity (D) and joining (J) gene segments to make a complete VH region and the recombination of variable and joining gene segments to make a complete VL region. The recombination process itself is imprecise, resulting in the loss or addition of amino acids at the V (D) J junctions. These mechanisms of diversity occur in the developing B cell prior to antigen exposure. After antigenic stimulation, the expressed antibody genes in B cells undergo somatic mutation.

Based on the estimated number of germline gene segments, the random recombination of these segments, and random VH-VL pairing, up to 1.6×107 different antibodies could be produced (Fundamental Immunology, 3rd ed., ed. Paul, Raven Press, New York, N.Y., 1993). When other processes which contribute to antibody diversity (such as somatic mutation) are taken into account, it is thought that upwards of 1×1010 different antibodies could be potentially generated (Immunoglobulin Genes, 2nd ed., eds. Jonio et al., Academic Press, San Diego, Calif., 1995). Because of the many processes involved in antibody diversity, it is highly unlikely that independently generated antibodies will have identical amino acid sequences in the CDRs.

The structure for carrying a CDR will generally be an antibody heavy or light chain or a portion thereof, in which the CDR is located at a location corresponding to the CDR of naturally occurring VH and VL. The structures and locations of immunoglobulin variable domains may be determined, for example, as described in Kabat et al., Sequences of Proteins of Immunological Interest, No. 91-3242, National Institutes of Health Publications, Bethesda, Md., 1991.

Amino acid sequences of VH and VL domains of humanized anti-KLRG1 antibodies are set forth in the Sequence Listing.

Anti-KLRG1 antibodies may optionally comprise antibody constant regions or parts thereof. For example, a VL domain may have attached, at its C terminus, antibody light chain constant domains including human CK or CA chains. Similarly, a specific antigen-binding domain based on a VH domain may have attached all or part of an immunoglobulin heavy chain derived from any antibody isotope, e.g., IgG, IgA, IgE, and IgM and any of the isotope sub-classes, which include but are not limited to, IgG1 and IgG4. In the exemplary embodiments, HG1N01, HG1N02, HG1N07, PA-015, and GA-015 antibodies comprise C-terminal fragments of heavy and light chains of human IgG1) or IgG1K. The DNA and amino acid sequences for the C-terminal fragment of are well known in the art (see, e.g., Kabat et al., Sequences of Proteins of Immunological Interest, No. 91-3242, National Institutes of Health Publications, Bethesda, Md., 1991).

In certain embodiments, the antibody or antigen-binding fragment can comprise a VH and/or VL domain of an Fv fragment from HG1N01, HG1N02, HG1N07, PA-015, or GA-015. Further embodiments comprise at least one CDR of any of these VH and VL domains. The CDRs can be defined by any appropriate antibody variable chain numbering scheme known to a person of ordinary skill in the art, such as those of Chothia, Kabat and Wu, AbM, Contact, and IMGT. The Sequence Listing table herein uses the numbering scheme of Chothia.

In certain embodiments, the VH and/or VL domains can be germlined, i.e., the framework regions (FRs) of these domains are mutated using conventional molecular biology techniques to match those produced by the germline cells. In other embodiments, the framework sequences remain diverged from the consensus germline sequences.

In certain embodiments, the antibodies or antigen-binding fragments can specifically bind an epitope within the ECD of human or mouse KLRG1, with an affinity, as expressed in KD, of at least about 2 nM, 1 nm, 100 pM, 10 pM, or 5 pM. The amino acid sequences of ECDs of human and cynomolgus KLRG1 are known in the art and need not be described.

It is contemplated that antibodies and antigen-binding fragments useful in the methods disclosed herein can also bind with other proteins, including, for example, recombinant proteins comprising all or a portion of KLRG1.

One of ordinary skill in the art will recognize that the antibodies and antigen-binding fragments useful in the methods disclosed herein may be used to detect, measure, and inhibit proteins that differ somewhat from KLRG1. The antibodies are expected to retain the specificity of binding so long as the target protein comprises a sequence which is at least about 60%, 70%, 80%, 90%, 91%, 92%, 93%, 94%, 95%, 96%, 97%, 98%, or 99% identical to any sequence of at least 130, 100, 80, 60, 40, or 20 contiguous amino acids in the sequence of wild type human or cynomolgus KLRG1. The percent identity can be determined by standard alignment algorithms such as, for example, Basic Local Alignment Tool (BLAST) described in Altshul et al. (1990) J. Mol. Biol., 215:403-410, the algorithm of Needleman et al. (1970) J. Mol. Biol., 48:444-453, or the algorithm of Meyers et al. (1988) Comput. Appl. Biosci., 4:11-17.

In addition to sequence homology analyses, epitope mapping (see, e.g., Epitope Mapping Protocols, ed. Morris, Humana Press, 1996) and secondary and tertiary structure analyses can be carried out to identify specific 3D structures assumed by the disclosed antibodies and antigen-binding fragments, and their complexes with antigens. Such methods include, but are not limited to, X-ray crystallography (Engstom (1974) Biochem. Exp. Biol., 11:7-13) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

The methods disclosed herein can also be performed with antibodies and antigen-binding fragments specific for KLRG1 other than those with sequences set forth in the Sequence Listing table herein. CDRs in such antibodies are not limited to the specific sequences of VH and VL identified in the Sequence Listing table, and may include variants of these sequences that retain the ability to specifically bind KLRG1. Such variants may be derived from the sequences listed in the Sequence Listing table by a skilled artisan using techniques well known in the art. For example, amino acid substitutions, deletions, or additions can be made in the FRs and/or in the CDRs. Variants of FRs also include naturally occurring immunoglobulin allotypes.

While changes in the FRs are usually designed to improve stability and immunogenicity of the antibody, changes in the CDRs are typically designed to increase affinity of the antibody for its target. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described in Antibody Engineering, 2nd ed., Oxford University Press, ed. Borrebaeck, 1995. These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs (see Table 1). Furthermore, any native residue in the polypeptide may also be substituted with alanine (see, e.g., MacLennan et al. (1998) Acta Physiol. Scand. Suppl. 643:55-67; Sasaki et al. (1998) Adv. Biophys. 35:1-24).

TABLE 1
Exemplary conservative substitutions:

| Residue | Conservative substitution |
|---|---|
| Ala (A) | Ser (S) |
| Arg (R) | Lys (K) |
| Asn (N) | Gln (Q); His (H) |
| Asp (D) | Glu (E) |
| Cys (C) | Ser (S) |
| Gln (Q) | Asn (N) |
| Glu (E) | Asp (D) |
| Gly (G) | Pro (P) |
| His (H) | Asn (N), Gln (Q) |
| Ile (I) | Leu (L), Val (V) |
| Leu (L) | Ile (I), Val (V) |
| Lys (K) | Arg (R), Gln (Q) |
| Met (M) | Leu (L), Ile (I) |
| Phe (F) | Met (M), Leu (L), Tyr (Y) |
| Ser (S) | Thr (T); Gly (G) |
| Thr (T) | Ser (S), Val (V) |
| Trp (W) | Tyr (Y) |
| Tyr (Y) | Trp (W), Phe (F) |
| Val (V) | Ile (I), Leu (L) |
| Pro (P) | — |

Derivatives and analogs of antibodies and antigen-binding fragments having sequences set forth in the Sequence Listing can be produced by various techniques well known in the art, including recombinant and synthetic methods (Maniatis (2012) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany).

In some embodiments, the antibody or the antigen-binding fragment can comprise a CDR-H1 selected from the group consisting of SEQ ID NO: 9, SEQ ID NO: 1, and SEQ ID NO: 16.

In some embodiments, the antibody or the antigen-binding fragment can comprise a CDR-H2 selected from the group consisting of SEQ ID NO: 10, SEQ ID NO: 2, and SEQ ID NO: 17.

In some embodiments, the antibody or the antigen-binding fragment can comprise a CDR-H3 selected from the group consisting of SEQ ID NO: 11, SEQ ID NO: 3, and SEQ ID NO: 18.

In some embodiments, the antibody or the antigen-binding fragment can comprise a CDR-L1 selected from the group consisting of SEQ ID NO: 12, SEQ ID NO:28, SEQ ID NO: 4, and SEQ ID NO: 19.

In some embodiments, the antibody or the antigen-binding fragment can comprise a CDR-L2 selected from the group consisting of SEQ ID NO: 5 and SEQ ID NO: 20.

In some embodiments, the antibody or the antigen-binding fragment can comprise a CDR-L3 selected from the group consisting of SEQ ID NO: 13, SEQ ID NO: 6, and SEQ ID NO: 21.

In some embodiments, the antibody or the antigen-binding fragment can comprise the CDR-H1 of SEQ ID NO: 9, the CDR-H2 of SEQ ID NO: 10, the CDR-H3 of SEQ ID NO: 11, the CDR-L1 of SEQ ID NO: 12, the CDR-L2 of SEQ ID NO: 5, and the CDR-L3 of SEQ ID NO: 13.

In some embodiments, the antibody or the antigen-binding fragment can comprise the CDR-H1 of SEQ ID NO: 9, the CDR-H2 of SEQ ID NO: 10, the CDR-H3 of SEQ ID NO: 11, the CDR-L1 of SEQ ID NO: 28, the CDR-L2 of SEQ ID NO: 5, and the CDR-L3 of SEQ ID NO: 13.

In some embodiments, the antibody or the antigen-binding fragment can comprise the CDR-H1 of SEQ ID NO: 1, the CDR-H2 of SEQ ID NO: 2, the CDR-H3 of SEQ ID NO: 3, the CDR-L1 of SEQ ID NO: 4, the CDR-L2 of SEQ ID NO: 5, and the CDR-L3 of SEQ ID NO: 6.

In some embodiments, the antibody or the antigen-binding fragment can comprise the CDR-H1 of SEQ ID NO: 16, the CDR-H2 of SEQ ID NO: 17, the CDR-H3 of SEQ ID NO: 18, the CDR-L1 of SEQ ID NO: 19, the CDR-L2 of SEQ ID NO: 20, and the CDR-L3 of SEQ ID NO: 21.

In some embodiments, the antibody or the antigen-binding fragment can comprise the CDR-H1 of SEQ ID NO: 9, the CDR-H2 of SEQ ID NO: 10, the CDR-H3 of SEQ ID NO: 11, the CDR-L1 of SEQ ID NO: 24, the CDR-L2 of SEQ ID NO: 5, and the CDR-L3 of SEQ ID NO: 13.

In some embodiments, the antibody or the antigen-binding fragment can comprise the CDR-H1 of SEQ ID NO: 9, the CDR-H2 of SEQ ID NO: 10, the CDR-H3 of SEQ ID NO: 11, the CDR-L1 of SEQ ID NO: 26, the CDR-L2 of SEQ ID NO: 5, and the CDR-L3 of SEQ ID NO: 6.

In some embodiments, the antibody or the antigen-binding fragment can comprise a heavy chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 14 and a light chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 15.

In some embodiments, the antibody or the antigen-binding fragment can comprise a heavy chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 7 and a light chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 8.

In some embodiments, the antibody or the antigen-binding fragment can comprise a heavy chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 22 and a light chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 23.

In some embodiments, the antibody or the antigen-binding fragment can comprise a heavy chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 24 and a light chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 25.

In some embodiments, the antibody or the antigen-binding fragment can comprise a heavy chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 14 and a light chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 27.

In some embodiments, the antibody or the antigen-binding fragment can comprise a heavy chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 14 and a light chain variable region with at least 90% identity, such as at least 91% identity, at least 92% identity, at least 93% identity, at least 94% identity, at least 95% identity, at least 96% identity, at least 97% identity, at least 98% identity, at least 99% identity, or 100% identity to SEQ ID NO: 29.

In some embodiments, the antibody or the antigen-binding fragment can comprise SEQ ID NO: 14 and SEQ ID NO: 15.

In some embodiments, wherein the N-terminal amino acid of an antibody sequence is a Q or an E, the disclosure provides for variants comprising cyclicized Q, i.e., pyro-E or pyroglutamate. Particularly, wherein the molecules of the disclosure are manufactured using bioprocessing, e.g., in a cell culture, cyclization (pyro-E) may be near 100% for Q. See, Liu et al., J Pharm Sci. 2019 October; 108 (10): 3194-3200 (PMID: 31145921) and Nguyen et al. (Int J Mol Sci. 2017 Jul. 20; 18 (7): 1575), which are incorporated by reference in parts pertinent thereto. As such, in some embodiments, the N-terminal amino acid residue of a VH chain disclosed herein, e.g., the VH chain having the amino acid sequence of SEQ ID NO: 7, 14, 22, or 24, is a pyro-glutamate. In one embodiment, the N-terminal “Q” residue of the amino acid sequence of SEQ ID NO:24 is a pyroglutamate.

### D. Nucleic Acid Agents as KLRG1 Antagonists

The anti-KLRG1 antibodies and antigen-binding fragments described herein can antagonize KLRG1 by binding to KLRG1 protein and interfering with its interaction with ligands such as cadherins. Another approach to reducing KLRG1 interaction with cadherins is to reduce the amount of KLRG1 protein present on the subject's cytotoxic T and NK cells. Nucleic acids that can suppress transcription, translation, or both of KLRG1 can reduce the amount of KLRG1 protein, and thus can be considered KLRG1 antagonists useful in methods of the present disclosure.

In some embodiments, the KLRG1 antagonist can be an antisense oligonucleotide (ASO). Antisense oligonucleotides (ASOs) are typically 15mer to 50mer DNA polynucleotides that comprise a sequence complementary to at least a target sequence of an RNA of interest, such as an RNA encoding KLRG1. Complementation of the RNA by the ASO may lead to destruction of the RNA, such as by the action of RNase H on the RNA. Destruction of the RNA may lead to reduced rates of translation and/or subsequent reduction in levels of the polypeptide product, in this case, KLRG1, which, having its translation rate and/or polypeptide product levels reduced, would lead to reduced activity and/or expression of KLRG1.

The ASO may comprise standard or chemically-modified DNA. Chemical modifications known in the art include, but are not limited to, phosphorothioate internucleoside linkages and modified bases, such as 2′-O-methoxy-ethyl (2′-MOE) bases and 5-methyl dC in CpG motifs. Such chemical modifications may increase resistance of the ASO to nucleases active against DNA, increase affinity of the ASO for the target sequence, reduce side effects of the ASO therapy, and/or impart other desirable properties to the ASO as will be known to a person of ordinary skill in the art.

In some embodiments, the KLRG1 antagonist can be an RNA interference (RNAi) agent. RNAi generally involves the introduction into cells of double stranded RNA (dsRNA) or short hairpin RNA (shRNA). Although the introduced RNAs may be of any length, a range of 300-600 nucleotides (nt) may be effective. Enzymes process these RNAs into short interfering RNA (siRNA) molecules with length about 21 nt, which yield single stranded RNA (ssRNA) which are incorporable into an RNA-induced silencing complex (RISC). The RISC includes endonucleases which may cleave a target mRNA complementary to the ssRNA. Alternatively, or in addition, and especially if the introduced RNA is incompletely complementary to the target mRNA, the target mRNA may form a dsRNA with the ssRNA, wherein the dsRNA cannot be translated into a polypeptide.

Variations of RNAi processes may include introducing siRNAs rather than longer RNAs requiring additional processing, and introducing vectors that express dsRNA, shRNA, or siRNA, among others known to a person of ordinary skill in the art.

Regardless of which mechanism(s) occur for a particular combination of introduced RNA and target mRNA, translation of the target mRNA may be reduced, thereby lowering levels of the polypeptide encoded by the target mRNA. Hence, RNAi therapy against RNA encoding the KLRG1 may reduce levels of KLRG1.

### E. Pharmaceutical Compositions

KLRG1 antagonists as described herein can be administered to the subject as a composition suitable for pharmaceutical use and administration. The compositions typically comprise one or more KLRG1 antagonists and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. (A specific class of active compounds, namely, vaccines against infectious agents, will be described in more detail herein). The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes, or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars; polyalcohols such as mannitol, sorbitol, and sodium chloride in the composition. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

Oral compositions generally include an inert diluent or an edible carrier. They can be enclosed in gelatin capsules or compressed into tablets. For oral administration, the KLRG1 antagonists can be combined with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers, or suppositories. For example, in some embodiments, wherein the KLRG1 antagonists are antibodies that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the KLRG1 antagonists may be delivered in the form of an aerosol spray from pressured container or dispenser, which contains a suitable propellant, e.g., a gas such as carbon dioxide, or a nebulizer.

In certain embodiments, the KLRG1 antagonists can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the KLRG1 antagonists can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

For any composition used in the present invention, the therapeutically effective dose can be estimated initially from cell culture assays. Examples of suitable bioassays include DNA replication assays, cytokine release assays, transcription-based assays, KLRG1/cadherin binding assays, immunological assays, or other assays as will be known to a person of ordinary skill in the art having the benefit of the present disclosure. The data obtained from the cell culture assays and animal studies can be used in formulating a range of dosage for use in humans. A dose may be formulated in animal models to achieve for antibodies or antigen-binding fragments a circulating plasma concentration range that includes the IC50 (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

### F. Non-KLRG1-Antagonist Therapies

In some embodiments, the method can further comprise administering to the subject an effective amount of a non-KLRG1-antagonist therapy for the infectious disease. The non-KLRG1-antagonist therapy can be any therapy other than an anti-KLRG1 antibody or antigen-binding fragment or a nucleic acid that can suppress transcription, translation, or both of KLRG1.

Examples of non-KLRG1-antagonist therapies include antiviral compounds, antibacterial compounds, antifungal compounds, antiparasitic compounds, and vaccines, among others that will be known to a person of ordinary skill in the art.

In some embodiments, the infectious disease can be tuberculosis and the non-KLRG1-antagonist therapy can be selected from the group consisting of isoniazid, rifampin, ethambutol, pyrazinamide, fluoroquinolones, amikacin, capreomycin, bedaquiline, linezolid, and combinations thereof.

In some embodiments, the infectious disease can be hepatitis B and the non-KLRG1-antagonist therapy can be selected from the group consisting of entecavir, tenofovir, lamivudine, adefovir, telbivudine, interferon alfa-2b, liver transplantation, and combinations thereof.

In some embodiments, the infectious disease can be chickenpox or shingles and the non-KLRG1-antagonist therapy can be acyclovir, valacyclovir, famciclovir, and combinations thereof.

In some embodiments, the infectious disease can be AIDS and the non-KLRG1-antagonist therapy can be selected from the group consisting of efavirenz, rilpivirine, doravirine, abacavir, tenofovir disoproxil fumarate, emtricitabine, lamivudine, zidovudine, emtricitabine/tenofovir disoproxil fumarate, emtricitabine/tenofovir alafenamide fumarate, atazanavir, darunavir, lopinavir/ritonavir, bictegravir sodium/emtricitabine/tenofovir alafenamide fumarate, raltegravir, dolutegravir, cabotegravir, enfuvirtide, maraviroc, and combinations thereof.

Notwithstanding the foregoing illustrative embodiments, one skilled in the art will appreciate that the KLRG1 antagonist can be combined with other non-KLRG1-antagonist therapies.

### G. Vaccine Therapies

In some embodiments, a non-KLRG1-antagonist therapy can be a vaccine therapy, i.e., the method can further comprise administering to the subject an effective amount of a vaccine therapy. As used herein, a vaccine therapy is an agent that stimulates an immune system response for preventing or treating infectious disease. Vaccine therapies are well-known, and include live microorganisms, attenuated microorganisms, bacille Calmette-Guerin (BCG), one or more cell surface proteins or viral coat proteins, and mRNA encoding one or more proteins, among others. Examples of approved and commercially available vaccine therapies include adjuvanted recombinant zoster vaccine (Shingrix, GSK plc, Brentford, United Kingdom) for herpes zoster (shingles). Other vaccine therapies are under development and in clinical trials.

In some embodiments, the infectious disease can be tuberculosis and the vaccine therapy can be bacille Calmette-Guerin (BCG).

In some embodiments, the infectious disease can be hepatitis B and the vaccine can be a recombinant hepatitis B vaccine. Exemplary recombinant hepatitis B vaccines include Engerix-B (GSK plc) and Recombivax-HB (Merck and Co., Inc., Rahway, NJ).

In some embodiments, the infectious disease can be chickenpox and the vaccine therapy can be a varicella vaccine.

In some embodiments, the infectious disease can be shingles and the vaccine therapy can be an adjuvanted recombinant zoster vaccine, such as Shingrix (GSK plc), or a live zoster vaccine, such as Zostavax (Merck and Co., Inc.).

The vaccine therapy and the KLRG1 antagonist can be administered to the subject essentially simultaneously (e.g., concurrently, during the same day, during the same course of treatment) or within a time period where the vaccine therapy and the KLRG1 antagonist combine favorably to achieve a desired effect (e.g., effective treatment, synergy). In some embodiments, the KLRG1 antagonist can allow for a more effective T cell response to the vaccine. In various embodiments, the KLRG1 antagonist and vaccine therapy can be synergistic. Synergy can be defined as the combination having greater than an additive (or otherwise expected) effect, which can be measured by various methods known in the art.

In various embodiments, the vaccine therapy can be Zoster Vaccine Live (Zostavax®). Without limitation, in such embodiments, the infectious disease can be herpes zoster (shingles).

In various embodiments, the vaccine therapy can be bacille Calmette-Guerin (BCG). Without limitation, in such embodiments, the infectious disease can be tuberculosis.

In various embodiments, the vaccine therapy can be Engerix-B (GSK) or Recombivax-HB (Merck and Co.). Without limitation, in such embodiments, the infectious disease can be hepatitis B.

Notwithstanding the foregoing illustrative embodiments, one skilled in the art will appreciate that the KLRG1 antagonist can be combined with other vaccine therapies.

H. Sequences

| | | SEQ | |
|---|---|---|---|
| Antibody | Region | ID NO: | Sequence |
| HG1N01 | CDR-H1 | 1 | GFSLTTFGM |
| HG1N01 | CDR-H2 | 2 | WWNDD |
| HG1N01 | CDR-H3 | 3 | TIYYGNYLTFYAMEH |
| HG1N01 | CDR-L1 | 4 | RSSQNIVHSNGNTYLE |
| HG1N01 | CDR-L2 | 5 | KVSNRFS |
| HG1N01 | CDR-L3 | 6 | FQGSHVPPT |
| HG1N01 | VH | 7 | QVILKESGPGLVKPTQ |
| | | | TLTLTCTFSGFSLTTF |
| | | | GMGIGWIRQPPGKALE |
| | | | WLAHIWWNDDKSYNSA |
| | | | LKSRLTISKDTSKNQV |
| | | | VLTMTNMDPVDTATYY |
| | | | CARTIYYGNYLTFYAM |
| | | | EHWGQGTTVTVSS |
| HG1N01 | VL | 8 | DILMTQSPLSLPVTPG |
| | | | EPASISCRSSQNIVHS |
| | | | NGNTYLEWYLQKPGQS |
| | | | PRLLIYKVSNRFSGVP |
| | | | DRFSGSGSGTDFTLKI |
| | | | SRVEAEDVGVYYCFQG |
| | | | SHVPPTFGAGTKLELK |
| | | | RTV |
| HG1N02 | CDR-H1 | 9 | GFSLSTFGM |
| HG1N02 | CDR-H2 | 10 | WWDDD |
| HG1N02 | CDR-H3 | 11 | VIYYGSRSAYYSMDY |
| HG1N02 | CDR-L1 | 12 | KSSQSIVHSNAHTYLE |
| HG1N02 | CDR-L1 | 28 | KSSQSIVHSNGHTYLE |
| | alt | | |
| HG1N02 | CDR-L2 | 5 | KVSNRFS |
| HG1N02 | CDR-L3 | 13 | FQGSHVPVT |
| HG1N02 | VH | 14 | QVTLKESGPGLVKPTQ |
| | | | TLTLTCTFSGFSLSTF |
| | | | GMGVGWIRQPPGKALE |
| | | | WLAHIWWDDDKWYELA |
| | | | LKSRLTISKDSSKNQV |
| | | | VLTMTNMDPVDTATYY |
| | | | CARVIYYGSRSAYYSM |
| | | | DYWGPGTTVTVSS |
| HG1N02 | VL | 15 | DILMTQSPLSLPVTPG |
| | | | EPASISCKSSQSIVHS |
| | | | NAHTYLEWYLQKPGQS |
| | | | PRLLIYKVSNRFSGVP |
| | | | DRFSGSGSGTDFTLKI |
| | | | SRVEAEDVGVYYCFQG |
| | | | SHVPVTFGAGTKLELK |
| | | | RTV |
| HG1N02 | VL alt | 29 | DILMTQSPLSLPVTPG |
| | | | EPASISCKSSQSIVHS |
| | | | NGHTYLEWYLQKPGQS |
| | | | PRLLIYKVSNRFSGVP |
| | | | DRFSGSGSGTDFTLKI |
| | | | SRVEAEDVGVYYCFQG |
| | | | SHVPVTFGAGTKLELK |
| | | | RTV |
| HG1N07 | CDR-H1 | 16 | GFTFRNY |
| HG1N07 | CDR-H2 | 17 | SESGNY |
| HG1N07 | CDR-H3 | 18 | DDWEGRAMDY |
| HG1N07 | CDR-L1 | 19 | RASRDIGSSLN |
| HG1N07 | CDR-L2 | 20 | ATSSLDS |
| HG1N07 | CDR-L3 | 21 | LQYASSPWT |
| HG1N07 | VH | 22 | QVQLVESGGGLVKPGG |
| | | | SLRLSCAASGFTFRNY |
| | | | AMSWIRQTPGKGLEWV |
| | | | ATISESGNYNNYPDNV |
| | | | KGRLTISRDNAKNSLY |
| | | | LQMNSLKAEDTAVYYC |
| | | | VRDDWEGRAMDYWGQG |
| | | | TTVTVSS |
| HG1N07 | VL | 23 | DIQMTQSPSSLSASVG |
| | | | DRVTITCRASRDIGSS |
| | | | LNWYQQKPGGAPKRLI |
| | | | YATSSLDSGVPKRFSG |
| | | | SGSGTDFTLTISSLQS |
| | | | EDFATYYCLQYASSPW |
| | | | TFGQGTKVEIKRTV |
| PA-015 | CDR-H1 | 9 | GFSLSTFGM |
| PA-015 | CDR-H2 | 10 | WWDDD |
| PA-015 | CDR-H3 | 11 | VIYYGSRSAYYSMDY |
| PA-015 | CDR-L1 | 12 | KSSQSIVHSNAHTYLE |
| PA-015 | CDR-L2 | 5 | KVSNRFS |
| PA-015 | CDR-L3 | 13 | FQGSHVPVT |
| PA-015 | VH | 24 | QVTLRESGPALVKPTQ |
| | | | TLTLTCTVSGFSLSTF |
| | | | GMGVGWIRQPPGKALE |
| | | | WLAHIWWDDDKWYELA |
| | | | LKSRLTISKDTSKNQV |
| | | | VLTITNMDPVDTATYY |
| | | | CARVIYYGSRSAYYSM |
| | | | DYWGQGTTVTVSS |
| PA-015 | VL | 25 | DVVMTQTPLSLSVTPG |
| | | | QPASISCKSSQSIVHS |
| | | | NAHTYLEWYLQKPGQS |
| | | | PQLLIYKVSNRFSGVP |
| | | | DRFSGSGSGTDFTLKI |
| | | | SRVEAEDVGVYYCFQG |
| | | | SHVPVTFGQGTKLEIK |
| | | | RTV |
| GA-015 | CDR-H1 | 9 | GFSLSTFGM |
| GA-015 | CDR-H2 | 10 | WWDDD |
| GA-015 | CDR-H3 | 11 | VIYYGSRSAYYSMDY |
| GA-015 | CDR-L1 | 26 | RSSQNIVHSNANTYLE |
| GA-015 | CDR-L2 | 5 | KVSNRFS |
| GA-015 | CDR-L3 | 6 | FQGSHVPPT |
| GA-015 | VH | 14 | QVTLKESGPGLVKPTQ |
| | | | TLTLTCTFSGFSLSTF |
| | | | GMGVGWIRQPPGKALE |
| | | | WLAHIWWDDDKWYELA |
| | | | LKSRLTISKDSSKNQV |
| | | | VLTMTNMDPVDTATYY |
| | | | CARVIYYGSRSAYYSM |
| | | | DYWGPGTTVTVSS |
| GA-015 | VL | 27 | DILMTQSPLSLPVTPG |
| | | | EPASISCRSSQNIVHS |
| | | | NANTYLEWYLQKPGQS |
| | | | PRLLIYKVSNRFSGVP |
| | | | DRFSGSGSGTDFTLKI |
| | | | SRVEAEDVGVYYCFQG |
| | | | SHVPPTFGAGTKLELK |
| | | | RTV |

### NUMBERED EMBODIMENTS

In addition to the sections outlined above, the methods of the present disclosure are also captured in the following enumerated embodiments:
1. A method of preventing a human immunodeficiency virus (HIV) infection in a subject or treating a subject suffering from an infectious disease caused by HIV, comprising administering to the subject an effective amount of an antibody or an antigen-binding fragment thereof that
(i) specifically binds killer cell lectin-like receptor G1 (KLRG1);
and
(ii) comprises a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 12, a CDR-L2 comprising the sequence of SEQ ID NO: 5, and a CDR-L3 comprising the sequence of SEQ ID NO: 13;
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 28, a CDR-L2 comprising the sequence of SEQ ID NO: 5, and a CDR-L3 comprising the sequence of SEQ ID NO: 13;
a CDR-H1 comprising the sequence of SEQ ID NO: 1, a CDR-H2 comprising the sequence of SEQ ID NO: 2, a CDR-H3 comprising the sequence of SEQ ID NO: 3, a CDR-L1 comprising the sequence of SEQ ID NO: 4, a CDR-L2 comprising the sequence of SEQ ID NO: 5, and a CDR-L3 comprising the sequence of SEQ ID NO: 6;
a CDR-H1 comprising the sequence of SEQ ID NO: 16, a CDR-H2 comprising the sequence of SEQ ID NO: 17, a CDR-H3 comprising the sequence of SEQ ID NO: 18, a CDR-L1 comprising the sequence of SEQ ID NO: 19, a CDR-L2 comprising the sequence of SEQ ID NO: 20, and a CDR-L3 comprising the sequence of SEQ ID NO: 21; or
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 26, a CDR-L2 comprising the sequence of SEQ ID NO: 5, and a CDR-L3 comprising the sequence of SEQ ID NO: 6.
2. The method of embodiment 1, wherein the CDR-H1 comprises the sequence of SEQ ID NO: 9, the CDR-H2 comprises the sequence of SEQ ID NO: 10, the CDR-H3 comprises the sequence of SEQ ID NO: 11, the CDR-L1 comprises the sequence of SEQ ID NO: 12, the CDR-L2 comprises the sequence of SEQ ID NO: 5, and the CDR-L3 comprises the sequence of SEQ ID NO: 13.
3. The method of embodiment 1 or 2, wherein the antibody or the antigen-binding fragment comprises
a heavy chain variable region with at least 90% identity to the sequence of SEQ ID NO: 24 and a light chain variable region with at least 90% identity to the sequence of SEQ ID NO: 25;
a heavy chain variable region with at least 90% identity to the sequence of SEQ ID NO: 14 and a light chain variable region with at least 90% identity to the sequence of SEQ ID NO: 15;
a heavy chain variable region with at least 90% identity to the sequence of SEQ ID NO: 14 and a light chain variable region with at least 90% identity to the sequence of SEQ ID NO: 29;
a heavy chain variable region with at least 90% identity to the sequence of SEQ ID NO: 7 and a light chain variable region with at least 90% identity to the sequence of SEQ ID NO: 8;
a heavy chain variable region with at least 90% identity to the sequence of SEQ ID NO: 22 and a light chain variable region with at least 90% identity to the sequence of SEQ ID NO: 23; or
a heavy chain variable region with at least 90% identity to the sequence of SEQ ID NO: 14 and a light chain variable region with at least 90% identity to the sequence of SEQ ID NO: 27.
4. The method of embodiment 3, wherein the antibody or the antigen-binding fragment comprises the sequences of SEQ ID NO: 24 and SEQ ID NO: 25.
5. A method of treating a subject suffering from AIDS, comprising administering to the subject an effective amount of an antibody or an antigen-binding fragment thereof that
(i) specifically binds killer cell lectin-like receptor G1 (KLRG1);
and
(ii) comprises a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 12, a CDR-L2 comprising the sequence of SEQ ID NO: 5, and a CDR-L3 comprising the sequence of SEQ ID NO: 13;
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 28, a CDR-L2 comprising the sequence of SEQ ID NO: 5, and a CDR-L3 comprising the sequence of SEQ ID NO: 13;
a CDR-H1 comprising the sequence of SEQ ID NO: 1, a CDR-H2 comprising the sequence of SEQ ID NO: 2, a CDR-H3 comprising the sequence of SEQ ID NO: 3, a CDR-L1 comprising the sequence of SEQ ID NO: 4, a CDR-L2 comprising the sequence of SEQ ID NO: 5, and a CDR-L3 comprising the sequence of SEQ ID NO: 6;
a CDR-H1 comprising the sequence of SEQ ID NO: 16, a CDR-H2 comprising the sequence of SEQ ID NO: 17, a CDR-H3 comprising the sequence of SEQ ID NO: 18, a CDR-L1 comprising the sequence of SEQ ID NO: 19, a CDR-L2 comprising the sequence of SEQ ID NO: 20, and a CDR-L3 comprising the sequence of SEQ ID NO: 21; or
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 26, a CDR-L2 comprising the sequence of SEQ ID NO: 5, and a CDR-L3 comprising the sequence of SEQ ID NO: 6.
6. The method of embodiment 5, wherein the CDR-H1 comprises the sequence of SEQ ID NO: 9, the CDR-H2 comprises the sequence of SEQ ID NO: 10, the CDR-H3 comprises the sequence of SEQ ID NO: 11, the CDR-L1 comprises the sequence of SEQ ID NO: 12, the CDR-L2 comprises the sequence of SEQ ID NO: 5, and the CDR-L3 comprises the sequence of SEQ ID NO: 13.
7. The method of embodiment 5 or 6, wherein the antibody or the antigen-binding fragment comprises
a heavy chain variable region with at least 90% identity to the sequence of SEQ ID NO: 24 and a light chain variable region with at least 90% identity to the sequence of SEQ ID NO: 25;
a heavy chain variable region with at least 90% identity to the sequence of SEQ ID NO: 14 and a light chain variable region with at least 90% identity to the sequence of SEQ ID NO: 15;
a heavy chain variable region with at least 90% identity to the sequence of SEQ ID NO: 14 and a light chain variable region with at least 90% identity to the sequence of SEQ ID NO: 29;
a heavy chain variable region with at least 90% identity to the sequence of SEQ ID NO: 7 and a light chain variable region with at least 90% identity to the sequence of SEQ ID NO: 8;
a heavy chain variable region with at least 90% identity to the sequence of SEQ ID NO: 22 and a light chain variable region with at least 90% identity to the sequence of SEQ ID NO: 23; or
a heavy chain variable region with at least 90% identity to the sequence of SEQ ID NO: 14 and a light chain variable region with at least 90% identity to the sequence of SEQ ID NO: 27.
8. The method of embodiment 7, wherein the antibody or the antigen-binding fragment comprises the sequences of SEQ ID NO: 24 and SEQ ID NO: 25.
9. A method of preventing an HIV infection in a subject or treating a subject suffering from an infectious disease caused by HIV, comprising administering to the subject an effective amount of a nucleic acid agent that suppresses transcription, translation, or both of KLRG1.
10. The method of embodiment 9, wherein the nucleic acid agent is an RNAi agent.
11. The method of embodiment 9, wherein the nucleic acid agent is an antisense oligonucleotide.
12. The method of any one of embodiments 1, 5, and 9, wherein the antibody or antigen-binding fragment thereof or the nucleic acid agent disrupts KLRG1 signaling with a ligand of KLRG1 selected from the group consisting of E-cadherin, N-cadherin, and R-cadherin.
13. The method of any one of embodiments 1-12, wherein the method prevents HIV infection in the subject.
14. The method of any one of embodiments 1-12, wherein the method treats HIV infection in the subject.
15. The method of any preceding embodiment, further comprising administering to the subject a non-KLRG1-antagonist therapy for HIV infection.
16. The method of embodiment 15, wherein the non-KLRG1-antagonist therapy is selected from the group consisting of efavirenz, rilpivirine, doravirine, abacavir, tenofovir disoproxil fumarate, emtricitabine, lamivudine, zidovudine, emtricitabine/tenofovir disoproxil fumarate, emtricitabine/tenofovir alafenamide fumarate, atazanavir, darunavir, lopinavir/ritonavir, bictegravir sodium/emtricitabine/tenofovir alafenamide fumarate, raltegravir, dolutegravir, cabotegravir, enfuvirtide, maraviroc, and combinations thereof.
17. The method of embodiment 15, wherein the non-KLRG1-antagonist therapy is a vaccine therapy.

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### EXAMPLES

The following are examples of methods and compositions of the invention. It is understood that various other embodiments may be practiced, given the general description provided herein.

### A. Example 1: Tuberculosis Treatment

FIG. 1 shows that PBMCs from patients with untreated tuberculosis compared to the same patients during successful treatment and to healthy people have increased expression of KLRG1 (2.98 fold ratio). KLRG1 expression decreases with treatment, indicating it is a biomarker associated with disease activity. Accordingly, tuberculosis is a particularly attractive target for therapies according to the present invention.

### B. Example 2: Tuberculosis Prevention

FIG. 2 shows that PBMCs from patients vaccinated with BCG vaccine against tuberculosis have increased expression of KLRG1 from day 0 (prior to vaccine) to day 168 (1.56 fold ratio). Accordingly, administration of a KLRG1 antagonist before, during, or after BCG vaccine administration is a particularly attractive target for therapies according to the present invention.

### C. Example 3: Tuberculosis Prevention

FIG. 3 shows that PBMCs from patients vaccinated with BCG vaccine against tuberculosis shows increased expression of KLRG1 from day 0 (prior to vaccine) to day 29 (1.93 fold ratio). The patients studied in Example 3 were a separate cohort from those studied in Example 2. FIG. 3 suggests administration before, during, or after BCG vaccine is a particularly attractive target for therapies according to the present invention.

### D. Example 4: Hepatitis B Treatment

FIG. 4 shows that CD8+ hepatitis B antigen-specific T cells from patients with chronic hepatitis B compared to patients with resolved hepatitis B and to patients with acute hepatitis B, prior to development of specific immune responses, shows increased expression of KLRG1 (3.73 fold ratio). KLRG1 expression decreases with disease resolution, indicating it is a biomarker associated with disease activity.

### E. Example 5: Epstein-Barr Virus (EBV) Treatment

FIG. 5 shows that antigen-specific (against an EBV antigen) CD8+ T cells show increased expression of KLRG1 (5.14 fold ratio).

### F. Example 6: Cytomegalovirus (CMV) Treatment

FIG. 6 shows that antigen-specific (against a CMV antigen) CD4+ T cells show increased expression of KLRG1 (8.18 fold ratio).

### G. Example 7: HIV Treatment

FIG. 7 shows that KLRG1 expression increases are associated with HIV progression. Data adapted from Streeck et al.

### REFERENCES

Blackburn S D, Shin H, Haining W N, Zou T, Workman C J, Polley A, et al. Coregulation of CD8+ T cell exhaustion by multiple inhibitory receptors during chronic viral infection. Nat Immunol. 2009; 10(1):29-37.
Cyktor J C, Carruthers B, Stromberg P, Flano E, Pircher H, Turner J. Killer cell lectin-like receptor G1 deficiency significantly enhances survival after Mycobacterium tuberculosis infection. Infect Immun. 2013; 81(4):1090-9.
Grundemann C, Schwartzkopff S, Koschella M, Schweier O, Peters C, Voehringer D, et al. The N K receptor KLRG1 is dispensable for virus-induced N K and CD8+ T-cell differentiation and function in vivo. Eur J Immunol. 2010; 40(5):1303-14.
Lázár-Molnár E, Chen B, Sweeney K A, Wang E J, Liu W, Lin J, Porcelli S A, Almo S C, Nathenson S G, Jacobs W R Jr. Programmed death-1 (PD-1)-deficient mice are extraordinarily sensitive to tuberculosis. Proc Natl Acad Sci USA. 2010; 27; 107(30):13402-7.
Shi L, Wang J M, Ren J P, Cheng Y Q, Ying R S, Wu X Y, et al. KLRG1 impairs CD4+ T cell responses via p16ink4a and p27kip1 pathways: role in hepatitis B vaccine failure in individuals with hepatitis C virus infection. J Immunol. 2014; 192(2):649-57.
Streeck H, Kwon D S, Pyo A, Flanders M, Chevalier M F, Law K, Jülg B, Trocha K, Jolin J S, Anahtar M N, Lian J, Toth I, Brumme Z, Chang J J, Caron T, Rodig S J, Milner D A Jr, Piechoka-Trocha A, Kaufmann D E, Walker B D, Altfeld M. Epithelial adhesion molecules can inhibit HIV-1-specific CD8⁺ T-cell functions. Blood. 2011; 117(19):5112-22.

What is claimed:

1. A method of preventing a human immunodeficiency virus (HIV) infection in a subject or treating a subject suffering from an infectious disease caused by HIV, comprising administering to the subject an effective amount of an antibody or an antigen-binding fragment thereof that
(i) specifically binds killer cell lectin-like receptor G1 (KLRG1);
and
(ii) comprises a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 12, a CDR-L2 comprising the sequence of SEQ ID NO: 5, and a CDR-L3 comprising the sequence of SEQ ID NO: 13;
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 28, a CDR-L2 comprising the sequence of SEQ ID NO: 5, and a CDR-L3 comprising the sequence of SEQ ID NO: 13;
a CDR-H1 comprising the sequence of SEQ ID NO: 1, a CDR-H2 comprising the sequence of SEQ ID NO: 2, a CDR-H3 comprising the sequence of SEQ ID NO: 3, a CDR-L1 comprising the sequence of SEQ ID NO: 4, a CDR-L2 comprising the sequence of SEQ ID NO: 5, and a CDR-L3 comprising the sequence of SEQ ID NO: 6;
a CDR-H1 comprising the sequence of SEQ ID NO: 16, a CDR-H2 comprising the sequence of SEQ ID NO: 17, a CDR-H3 comprising the sequence of SEQ ID NO: 18, a CDR-L1 comprising the sequence of SEQ ID NO: 19, a CDR-L2 comprising the sequence of SEQ ID NO: 20, and a CDR-L3 comprising the sequence of SEQ ID NO: 21; or
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 26, a CDR-L2 comprising the sequence of SEQ ID NO: 5, and a CDR-L3 comprising the sequence of SEQ ID NO: 6.

2. The method of claim 1, wherein the CDR-H1 comprises the sequence of SEQ ID NO: 9, the CDR-H2 comprises the sequence of SEQ ID NO: 10, the CDR-H3 comprises the sequence of SEQ ID NO: 11, the CDR-L1 comprises the sequence of SEQ ID NO: 12, the CDR-L2 comprises the sequence of SEQ ID NO: 5, and the CDR-L3 comprises the sequence of SEQ ID NO: 13.

3. The method of claim 1 or 2, wherein the antibody or the antigen-binding fragment comprises
a heavy chain variable region with at least 90% identity to the sequence of SEQ ID NO: 24 and a light chain variable region with at least 90% identity to the sequence of SEQ ID NO: 25;
a heavy chain variable region with at least 90% identity to the sequence of SEQ ID NO: 14 and a light chain variable region with at least 90% identity to the sequence of SEQ ID NO: 15;
a heavy chain variable region with at least 90% identity to the sequence of SEQ ID NO: 14 and a light chain variable region with at least 90% identity to the sequence of SEQ ID NO: 29;
a heavy chain variable region with at least 90% identity to the sequence of SEQ ID NO: 7 and a light chain variable region with at least 90% identity to the sequence of SEQ ID NO: 8;
a heavy chain variable region with at least 90% identity to the sequence of SEQ ID NO: 22 and a light chain variable region with at least 90% identity to the sequence of SEQ ID NO: 23; or
a heavy chain variable region with at least 90% identity to the sequence of SEQ ID NO: 14 and a light chain variable region with at least 90% identity to the sequence of SEQ ID NO: 27.

4. The method of claim 3, wherein the antibody or the antigen-binding fragment comprises the sequences of SEQ ID NO: 24 and SEQ ID NO: 25.

5. A method of treating a subject suffering from AIDS, comprising administering to the subject an effective amount of an antibody or an antigen-binding fragment thereof that
(i) specifically binds killer cell lectin-like receptor G1 (KLRG1);
and
(ii) comprises a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 12, a CDR-L2 comprising the sequence of SEQ ID NO: 5, and a CDR-L3 comprising the sequence of SEQ ID NO: 13;
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 28, a CDR-L2 comprising the sequence of SEQ ID NO: 5, and a CDR-L3 comprising the sequence of SEQ ID NO: 13;
a CDR-H1 comprising the sequence of SEQ ID NO: 1, a CDR-H2 comprising the sequence of SEQ ID NO: 2, a CDR-H3 comprising the sequence of SEQ ID NO: 3, a CDR-L1 comprising the sequence of SEQ ID NO: 4, a CDR-L2 comprising the sequence of SEQ ID NO: 5, and a CDR-L3 comprising the sequence of SEQ ID NO: 6;
a CDR-H1 comprising the sequence of SEQ ID NO: 16, a CDR-H2 comprising the sequence of SEQ ID NO: 17, a CDR-H3 comprising the sequence of SEQ ID NO: 18, a CDR-L1 comprising the sequence of SEQ ID NO: 19, a CDR-L2 comprising the sequence of SEQ ID NO: 20, and a CDR-L3 comprising the sequence of SEQ ID NO: 21; or
a CDR-H1 comprising the sequence of SEQ ID NO: 9, a CDR-H2 comprising the sequence of SEQ ID NO: 10, a CDR-H3 comprising the sequence of SEQ ID NO: 11, a CDR-L1 comprising the sequence of SEQ ID NO: 26, a CDR-L2 comprising the sequence of SEQ ID NO: 5, and a CDR-L3 comprising the sequence of SEQ ID NO: 6.

6. The method of claim 5, wherein the CDR-H1 comprises the sequence of SEQ ID NO: 9, the CDR-H2 comprises the sequence of SEQ ID NO: 10, the CDR-H3 comprises the sequence of SEQ ID NO: 11, the CDR-L1 comprises the sequence of SEQ ID NO: 12, the CDR-L2 comprises the sequence of SEQ ID NO: 5, and the CDR-L3 comprises the sequence of SEQ ID NO: 13.

7. The method of claim 5 or 6, wherein the antibody or the antigen-binding fragment comprises
a heavy chain variable region with at least 90% identity to the sequence of SEQ ID NO: 24 and a light chain variable region with at least 90% identity to the sequence of SEQ ID NO: 25;
a heavy chain variable region with at least 90% identity to the sequence of SEQ ID NO: 14 and a light chain variable region with at least 90% identity to the sequence of SEQ ID NO: 15;
a heavy chain variable region with at least 90% identity to the sequence of SEQ ID NO: 14 and a light chain variable region with at least 90% identity to the sequence of SEQ ID NO: 29;
a heavy chain variable region with at least 90% identity to the sequence of SEQ ID NO: 7 and a light chain variable region with at least 90% identity to the sequence of SEQ ID NO: 8;
a heavy chain variable region with at least 90% identity to the sequence of SEQ ID NO: 22 and a light chain variable region with at least 90% identity to the sequence of SEQ ID NO: 23; or
a heavy chain variable region with at least 90% identity to the sequence of SEQ ID NO: 14 and a light chain variable region with at least 90% identity to the sequence of SEQ ID NO: 27.

8. The method of claim 7, wherein the antibody or the antigen-binding fragment comprises the sequences of SEQ ID NO: 24 and SEQ ID NO: 25.

9. A method of preventing an HIV infection in a subject or treating a subject suffering from an infectious disease caused by HIV, comprising administering to the subject an effective amount of a nucleic acid agent that suppresses transcription, translation, or both of KLRG1.

10. The method of claim 9, wherein the nucleic acid agent is an RNAi agent.

11. The method of claim 9, wherein the nucleic acid agent is an antisense oligonucleotide.

12. The method of any one of claims 1, 5, and 9, wherein the antibody or antigen-binding fragment thereof or the nucleic acid agent disrupts KLRG1 signaling with a ligand of KLRG1 selected from the group consisting of E-cadherin, N-cadherin, and R-cadherin.

13. The method of any one of claims 1, 5, and 9, wherein the method prevents HIV infection in the subject.

14. The method of any one of claims 1, 5, and 9, wherein the method treats HIV infection in the subject.

15. The method of any one of claims 1, 5, and 9, further comprising administering to the subject a non-KLRG1-antagonist therapy for HIV infection.

16. The method of claim 15, wherein the non-KLRG1-antagonist therapy is selected from the group consisting of efavirenz, rilpivirine, doravirine, abacavir, tenofovir disoproxil fumarate, emtricitabine, lamivudine, zidovudine, emtricitabine/tenofovir disoproxil fumarate, emtricitabine/tenofovir alafenamide fumarate, atazanavir, darunavir, lopinavir/ritonavir, bictegravir sodium/emtricitabine/tenofovir alafenamide fumarate, raltegravir, dolutegravir, cabotegravir, enfuvirtide, maraviroc, and combinations thereof.

17. The method of claim 15, wherein the non-KLRG1-antagonist therapy is a vaccine therapy.


SEQUENCE LISTING (29 sequences)

SEQ ID NO: 1 - protein, 9 aa, synthetic construct
GFSLTTFGM

SEQ ID NO: 2 - protein, 5 aa, synthetic construct
WWNDD

SEQ ID NO: 3 - protein, 15 aa, synthetic construct
TIYYGNYLTFYAMEH

SEQ ID NO: 4 - protein, 16 aa, synthetic construct
RSSQNIVHSNGNTYLE

SEQ ID NO: 5 - protein, 7 aa, synthetic construct
KVSNRFS

SEQ ID NO: 6 - protein, 9 aa, synthetic construct
FQGSHVPPT

SEQ ID NO: 7 - protein, 125 aa, synthetic construct
QVILKESGPGLVKPTQTLTLTCTFSGFSLTTFGMGIGWIRQPPGKALEWLAHIWWNDDKSYNSALKSRLTISKDTSKNQVVLTMTNMDPVDTATYYCARTIYYGNYLTFYAMEHWGQGTTVTVSS

SEQ ID NO: 8 - protein, 115 aa, synthetic construct
DILMTQSPLSLPVTPGEPASISCRSSQNIVHSNGNTYLEWYLQKPGQSPRLLIYKVSNRFSGVPDRFSGSGSGTDFTLKISRVEAEDVGVYYCFQGSHVPPTFGAGTKLELKRTV

SEQ ID NO: 9 - protein, 9 aa, synthetic construct
GFSLSTFGM

SEQ ID NO: 10 - protein, 5 aa, synthetic construct
WWDDD

SEQ ID NO: 11 - protein, 15 aa, synthetic construct
VIYYGSRSAYYSMDY

SEQ ID NO: 12 - protein, 16 aa, synthetic construct
KSSQSIVHSNAHTYLE

SEQ ID NO: 13 - protein, 9 aa, synthetic construct
FQGSHVPVT

SEQ ID NO: 14 - protein, 125 aa, synthetic construct
QVTLKESGPGLVKPTQTLTLTCTFSGFSLSTFGMGVGWIRQPPGKALEWLAHIWWDDDKWYELALKSRLTISKDSSKNQVVLTMTNMDPVDTATYYCARVIYYGSRSAYYSMDYWGPGTTVTVSS

SEQ ID NO: 15 - protein, 115 aa, synthetic construct
DILMTQSPLSLPVTPGEPASISCKSSQSIVHSNAHTYLEWYLQKPGQSPRLLIYKVSNRFSGVPDRFSGSGSGTDFTLKISRVEAEDVGVYYCFQGSHVPVTFGAGTKLELKRTV

SEQ ID NO: 16 - protein, 7 aa, synthetic construct
GFTFRNY

SEQ ID NO: 17 - protein, 6 aa, synthetic construct
SESGNY

SEQ ID NO: 18 - protein, 10 aa, synthetic construct
DDWEGRAMDY

SEQ ID NO: 19 - protein, 11 aa, synthetic construct
RASRDIGSSLN

SEQ ID NO: 20 - protein, 7 aa, synthetic construct
ATSSLDS

SEQ ID NO: 21 - protein, 9 aa, synthetic construct
LQYASSPWT

SEQ ID NO: 22 - protein, 119 aa, synthetic construct
QVQLVESGGGLVKPGGSLRLSCAASGFTFRNYAMSWIRQTPGKGLEWVATISESGNYNNYPDNVKGRLTISRDNAKNSLYLQMNSLKAEDTAVYYCVRDDWEGRAMDYWGQGTTVTVSS

SEQ ID NO: 23 - protein, 110 aa, synthetic construct
DIQMTQSPSSLSASVGDRVTITCRASRDIGSSLNWYQQKPGGAPKRLIYATSSLDSGVPKRFSGSGSGTDFTLTISSLQSEDFATYYCLQYASSPWTFGQGTKVEIKRTV

SEQ ID NO: 24 - protein, 125 aa, synthetic construct
QVTLRESGPALVKPTQTLTLTCTVSGFSLSTFGMGVGWIRQPPGKALEWLAHIWWDDDKWYELALKSRLTISKDTSKNQVVLTITNMDPVDTATYYCARVIYYGSRSAYYSMDYWGQGTTVTVSS

SEQ ID NO: 25 - protein, 115 aa, synthetic construct
DVVMTQTPLSLSVTPGQPASISCKSSQSIVHSNAHTYLEWYLQKPGQSPQLLIYKVSNRFSGVPDRFSGSGSGTDFTLKISRVEAEDVGVYYCFQGSHVPVTFGQGTKLEIKRTV

SEQ ID NO: 26 - protein, 16 aa, synthetic construct
RSSQNIVHSNANTYLE

SEQ ID NO: 27 - protein, 115 aa, synthetic construct
DILMTQSPLSLPVTPGEPASISCRSSQNIVHSNANTYLEWYLQKPGQSPRLLIYKVSNRFSGVPDRFSGSGSGTDFTLKISRVEAEDVGVYYCFQGSHVPPTFGAGTKLELKRTV

SEQ ID NO: 28 - protein, 16 aa, synthetic construct
KSSQSIVHSNGHTYLE

SEQ ID NO: 29 - protein, 115 aa, synthetic construct
DILMTQSPLSLPVTPGEPASISCKSSQSIVHSNGHTYLEWYLQKPGQSPRLLIYKVSNRFSGVPDRFSGSGSGTDFTLKISRVEAEDVGVYYCFQGSHVPVTFGAGTKLELKRTV